SEQUENCE LISTING (10 sequences)

SEQ ID NO: 1 - DNA, 2450 bp, Zea mays
tattatacgtacgagccaagtttgaactcgcaagaaggaaagccgatatgatcgagtattccttggagtgaacatggcctcctctacaaattcacatgaaaaattcagaatggggcctaattggtttgtactagctagtttacagctttacagaggaaagaggcagggcggaaaaactaaaagtgctacttatcctagctagttttcatcatcacttattgacaaggccataccatcagcagtctcaaatccaaggtgaatacattcacacactaccgaaaaactccatgcatgcgcacgaagctaggttaatcaactgaacagtgtgtgaactccggttaaaattaaagtaaccagccttcaccgcgtaagtatgaacaggtaactgcatttaattactactgccctctattagtttgaaaccctctccagtagacctctctaggtttgacggtcaaagttttaggcgaggggagggggaggcccagctagcggtgcagttaccgagataaaaacacacaaggcccctatcttaacctccacttctcctccccccgtaaagagagattaaaaaaggattagttcttctgtactagcctggcgtccgagagagagagagcacccggccttgttaacaattcgtcctggaggatcctatgcgaggtttttagggtgttttttgctcacttctccatgcgcggcgttgaagctagacgatggacttgcttgggaacgccaagcttttctatggccggaagacaagggttcgtgtcctcttctaggatgcaggtggcacgtactctccgtccagcatgcatgaacttggtcctcgtcttttcatcttttctgcctgtcgagtgtcgacccttcaattcatcttttgaagcaaattatagtgtagtatttctttccctttttttttgaagcatgcattgcaagtagggattgtcgtgcaccatctacaatactccattgagagtattgactgactatcgattgcattgaaagaatttttcctatctcacacagtatcatatgcatttggatacagaccatttcattattttacgatagctagggtaggaattctttagttctagctaaatcttgatcatgactggagttttggaatgattgtgtgaaatattacacggtatatatataatgattgtagtacttgtgttagatggtgagataatggagctaggaagggcgaacctaggattttgtgaaaaacccatatcagatacatgttcagcttctgcatgtgactgtgaatgcgttagggttaatataattaaggagaggcctagaccataccaggagtaattgctcatatgcttatttcagcaggcttgaacagtgttttggctcgtagctggggcagcttgatgctgtaatatacagttgaagaagcgtatacatgcatgcatatgtcaactggatgtgcataaatgtccaggcactggtgtactgtacccgtgtgcgtgcatgtgcatctatcatacaattgaaaaaaaaaatcctaccagaacatctgcttactaattgagattctagacaaatagaacaaaacttagatctcgtgtcatttcttttttttccttcttccaagatgggttcgatttcttaatcgttcttgacagcaacctgccagtcaaatggccgtgacaacgtatactattatcgagtaaaaggtcgccactttagtagtacatgtacatgcatgcgcagatacatcatcaggtactcatatatgggcacacatatagacatgttttgaggaaaatgagacaaagtatagtggagacttccctagaaagcagaagaaaaagaagtggtttatgttccgttaaatcatactacaacttttttttattatactctccattttgtcatcattaggtactcatatatgggcacacatatagtactgccaatttttcttgctaaaaaaagttccactatatatatgtatgtatgcacaaataaactaattttcttagaaaagaaaaccggtgtaatacatactaagggctagtttgggaaccctggttttctaaggaattttatttttccaaaaaaaatagtttatttttccttcggaaattaggaatctcttataaaattcgagttcccaaactattcctaatatatatatcatactctccatcagtctatatatagattacatatagtaagtatagagtatctcgctatcacatagtgccactaatcttctggagtgtaccagttgtataaatatctatcagtatcagcactactgtttgctgaataccccaaaactctctgcttgacttctcttccctaacctttgcactgtccaaaatggcttcctgatcccctcacttcctcgaatcattctaagaagaaactcaagccgctaccattaggggcagattaatagcgctactttcagataatctacc

SEQ ID NO: 2 - DNA, 2358 bp, Zea mays
ttgaaaaattcagaatggggcctaattggtttgtactagctagtttacagctttacagaggaaagaggcagggcggaaaaactaaaagtgctgcttatcctagctagttttcatcatcacttattgacaaggccataccatcagcagtctcaaatccaaggtgaatacattcacacactaccgaaaaactccatgcatgcgcacgaagctaggttaatcaactgaacagtgtgtgaactccggttaaaattaaagtaaccagccttcaccgcgtaagtatgaacaggtaactgcatttaattactactgccctctattagtttgaaaccctctccagtagacctctctaggtttgacggtcaaagttttaggcgaggggagggggaggcccagctagcggtgcagttaccgagataaaaacacacaaggcccctatcttaacctccacttctcctccccccgtaaagagagattaaaaaaggattagttcttctgtactagcctggcgtccgagagagagagagagcacccggccttgttaacaattcgtcctggaggatcctatgcgaggtttttagggtgttttttgctcacttctccatgcgcggcgttgaagctagacgatggacttgcttgggaacgccaagcttttctatggccggaagacaagggttcgtgtcctcttctaggatgcaggtggcacgtactctccgtccagcatgcatgaacttggtcctcgtcttttcatcttttctgcctgtcgagtgtcgacccttcaattcatcttttgaagcaaattatagtgtagtatttctttccctttttttttgaagcatgcattgcaagtagggattgtcgtgcaccatctacaatactccattgagagtattgactgactatcgattgcattgaaagaatttttcctatctcacacagtatcatatgcatttggatacagtccatttcattattttacgatagctagggtaggaattctttagttctagctaaatcttgatcatgactggagttttggaatgattgtgtgaaatattacacggtatatatataatgattgtagtacttgtgttagatggtgagataatggagctaggaagggcgaacctaggattttgtgaaaaacccatatcagatacatgttcagcttctgcatgtgactgtgaatgcgttagggttaatataattaaggagaggcctagaccataccaggagtaattgctcatatgcttatttcagcaggcttgaacagtgttttggctcgtagctggggcagcttgatgctgtaatatacagttgaagaagcgtatacatgcatgcatatgtcaactggatgtgcataaatgtccaggcactggtgtactgtacccgtgtgcgtgcatgtgcatctatcatacaattgaaaaaaaaaatcctaccagaacatctgcttactaattgagattctagacaaatagaacaaaacttagatctcgtgtcatttcttttttttccttcttccaagatgggttcgatttcttaatcgttcttgacagcaacctgccagtcaaatggccgtgacaacgtatactattatcgagtaaaaggtcgccccctttagtagtacatgtacatgcatgcgcagatacatcatcaggtactcatatatgggcacacatatagacatgttttgaggaaaatgagacaaagtatagtggagacttccctagaaagcagaagaaaaagaagtggtttatgttccgttaaatcatactacaacttttttttattatactctccattttgtcaccattaggtactcatatatgggcacacatatagtactgccaatttttcttgctaaaaaaagttccactatatatatgtatgtatgcacaaataaactaattttcttagaaaagaaaaccggtgtaatacatactaagggctagtttgggaaccctggttttctaaggaattttatttttccaaaaaaaatagtttatttttctttcggaaattaggaatctcttataaaattcgagttcccaaactattcctaatatatatatcatactctccatcagtctatatatagattacatatagtaagtatagagtatctcgctatcacatagtgccactaatcttctggagtgtaccagttgtataaatatctatcagtatcagcactactgtttgctgaataccccaaaactctctgcttgacttctcttccctaacctttgcactgtccaaaatggcttcctgatcccctcacttcctcgaatcattctaagaagaaactcaagccgctaccattaggggcagattaatagcgctactttcagataatctacc

SEQ ID NO: 3 - DNA, 2354 bp, Zea mays
gaaaaattcagaatggggcctaattggtttgtactagctagtttacagctttacagaggaaagaggcagggcggaaaaactaaaagtgctacttatcctagctagttttcatcatcacttattgacaaggccataccatcagcagtctcaaatccaaggtgaatacattcacacactaccgaaaaactccatgcatgcgcacgaagctaggttaatcaactgaacagtgtgtgaactccggttaaaattaaagtaaccagccttcaccgcgtaagtatgaacaggtaactgcatttaattactactgccctctattagtttgaaaccctctccagtagacctctctaggtttgacggtcaaagttttaggcgaggggagggggaggcccagctagcggtgcagttaccgagataaaaacacacaaggcccctatcttaacctccacttctcctccccccgtaaagagagattaaaaaaggattagttcttctgtactagcctggcgtccgagagagagagagagcacccggccttgttaacaattcgtcctggaggatcctatgcgaggtttttagggtgttttttgctcacttctccatgcgcggcgttgaagctagacgatgggcttgcttgggaacgccaagcttttctatggccggaagacaagggttcgtgtcctcttctaggatgcaggtggcacgtactctccgtccagcatgcatggacttggtcctcgtcttttcatcttttctgcctgtcgagtgtcgacccttcaattcatcttttgaagcaaattatagtgtagtatttctttccctttttttttgaagcatgcattgcaagtagggattgtcgtgcaccatctacaatactccattgagagtattgactgactatcgattgcattgaaagaatttttcctatctcacacagtatcatatgcatttggatacagaccatttcattattttacgatagctagggtaggaattctttagttctagctaaatcttgatcatgactggagttttggaatgattgtgtgaaatattacacggtatatatataatgattgtagtacttgtgttagatggtgagataatggagctaggaagggcgaacctaggattttgtgaaaaacccatatcagatacatgttcagcttctgcatgtgactgtgaatgcgttagggttaatataattaaggagaggcctagaccataccaggagtaattgctcatatgcttatttcagcaggcttgaacagtgttttggctcgtagctggggcagcttgatgctgtaatatacagttgaagaagcgtatacatgcatgcatatgtcaactggatgtgcataaatgtccaggcactggtgtactgtacccgtgtgcgtgcatgtgcatctatcatacaattgaaaaaaaaaatcctaccagaacatctgcttactaattgagattctagacaaatagaacaaaacttagatctcgtgtcatttcttttttttccttcttccaagatgggttcgatttcttaatcgttcttgacagcaacctgccagtcaaatggccgtgacaacgtatactattatcgagtaaaaggtcgccactttagtagtacatgtacatgcatgcgcagatacatcatcaggtactcatatatgggcacacatatagacatgttttgaggaaaatgagacaaagtgtagtggagacttccctagaaagcagaagaaaaagaagtggtttatgttccgttaaatcatactacaacttttttttattatactctccattttgtcatcattaggtactcatatatgggcacacatatagtactgccaatttttcttgctaaaaaaagttccactatatatatgtatgtatgcacaaataaactaattttcttagaaaagaaaaccggtgtaatacatactaagggctagtttgggaaccctggttttctaaggaattttatttttccaaaaaaatagtttatttttccttcggaaattaggaatctcttataaaattcgagttcccaaactattcctaatatatatatcatactctccatcagtctatatatagattacatatagtaagtatagagtatctcgctatcacatagtgccactaatcttctggagtgtaccagttgtataaatatctatcagtatcagcactactgtttgctgaataccccaaaactctctgcttgacttctcttccctaacctttgcactgtccaaaatggcttcctgatcccctcacttcctcgaatcattctaagaagaaactcaagccgctaccattaggggcagattaatagcgctactttcagataatctacc

SEQ ID NO: 4 - DNA, 20 bp, Artificial Sequence
  Primer
tgtacatgcatgcgcagata

SEQ ID NO: 5 - DNA, 20 bp, Artificial Sequence
  Primer
caaactgtgctgctagtgct

SEQ ID NO: 6 - DNA, 20 bp, Artificial Sequence
  Primer
tttgaagcatgcattgcaag

SEQ ID NO: 7 - DNA, 20 bp, Artificial Sequence
  Primer
tgaaaaattcagaatggggc

SEQ ID NO: 8 - DNA, 40 bp, Artificial Sequence
  Primer
ggttacccggaccggagctctattatacgtacgagccaag

SEQ ID NO: 9 - DNA, 52 bp, Artificial Sequence
  Primer
ccatggtagattatctgaaagtagcgctattaatctgcccctaatggtagcg

SEQ ID NO: 10 - DNA, 40 bp, Artificial Sequence
  Primer
ggttacccggaccggaattcttagttctagctaaatcttg

(19) United States
(12) Patent Application Publication
(10) Pub. No.: US 2005/0223432 A1
(43) Pub. Date: Oct. 6, 2005

(54) ODP2 promoter and methods of use

(75) Inventors: Shane E. Abbitt, Ankeny, IA (US); William J. Gordon-Kamm, Urbandale, IA (US); Keith S. Lowe, Johnston, IA (US); Peizhong Zheng, Johnston, IA (US)

Correspondence Address: ALSTON & BIRD LLP;PIONEER HI-BRED INTERNATIONAL, INC., BANK OF AMERICA PLAZA, 101 SOUTH TYRON STREET, SUITE 4000, CHARLOTTE, NC, 28280-4000, US

(73) Assignee: Pioneer Hi-Bred International, Inc., Johnston, IA (US)

(21) Appl. No.: 11/000,752

(22) Filed: Dec. 1, 2004

Related U.S. Application Data
(60) Provisional application No. 60/541,171, filed on Feb. 2, 2004.

(51) Int. Cl. A01H 5/00; A01H 1/00; C12N 5/10; C12N 15/82; C12N 15/29

(52) U.S. Cl. 800/290; 536/23.6; 435/320.1; 435/419; 800/298; 800/320.1

(57) ABSTRACT

Compositions and methods for regulating expression of nucleotide sequences of interest in a plant are provided. Compositions include novel nucleic acid molecules, and variants and fragments thereof, for promoter sequences isolated from the maize Ovule Development Protein 2 (ODP2) gene. A method for expressing a nucleotide sequence of interest in a plant using the promoter sequences disclosed herein is further provided. The method comprises introducing into a plant or plant cell an expression cassette comprising an ODP2 promoter of the present invention operably linked to a nucleotide sequence of interest. In particular, the compositions and methods find use in regulating expression of nucleotide sequences of interest in a seed-preferred manner. Transformed plants, plant cells, and seeds comprising the ODP2 promoter sequence or variants and fragments thereof are also provided.

### CROSS-REFERENCE TO RELATED APPLICATIONS

This application claims priority to U.S. Provisional Application Ser. No. 60/541,171, filed Feb. 2, 2004, which is incorporated herein by reference in its entirety.

### FIELD OF THE INVENTION

The present invention relates to the field of plant molecular biology, more particularly to regulation of gene expression in plants.

### BACKGROUND OF THE INVENTION

Plants are frequently genetically engineered to display commercially and argonomically important traits. Isolated plant promoter sequences find use in genetically modifying plants by driving the expression of heterologous nucleotide sequences of interest in order to vary the phenotype of the plant. Generally, plant promoters may be constitutive and drive expression in essentially all tissues, or, alternatively, they may be tissue-preferred and regulate expression in a tissue-specific manner. Promoters appropriate for the expression of a particular gene of interest are selected based on when and in what tissues within the plant expression of the heterologus DNA is desired. Therefore, a variety of promoters is needed to generate transformed plants with useful traits.

Frequently it is desirable to express a nucleotide sequence of interest in particular tissues or organs of a plant. Constitutive expression of some heterologous proteins, such as insecticides, leads to undesirable phenotypic and argonomic effects. Tissue-preferred promoters that control the expression of genes of interest in a tissue-specific manner facilitate greater control over the location and timing of expression of heterologous DNA sequences and reduce the possibility of deleterious effects on overall plant growth. Thus, the identification of tissue-preferred promoters is needed.

Embryogenesis is a critical stage of the plant life cycle in which the overall architectural pattern of the mature plant is established. The root and shoot apical meristems are specified, thereby establishing the basic structure of the seedling. Differentiation of tissues and organs also occurs during embryogenesis. Given the importance of embryogenesis to the overall development of the mature plant, control of gene expression during this stage is of particular interest. Thus, plant promoters that could be used to preferentially drive expression of heterologous nucleotide sequences in the plant embryo or seed are desired.

Seed-preferred promoters could be used to preferentially express a variety of genes of interest in a plant, including, for example, those involved in regulation of embryo development, tissue differentiation, and biosynthesis of lipids, proteins, and carbohydrates. Other useful genes for seed-preferred expression include genes that confer plant resistance to a variety of environmental factors or that increase oil content in the seed. Therefore, the isolation and characterization of tissue-preferred, particularly seed-preferred, promoters that can direct transcription of a sufficiently high level of a desired heterologous nucleotide sequence are needed.

### BRIEF SUMMARY OF THE INVENTION

Compositions and methods for regulating gene expression in a plant are provided. Compositions include novel nucleotide sequences for promoters isolated from the maize ODP2 gene. The promoter sequences of the invention initiate transcription in a seed-preferred manner. More particularly, the compositions of the invention comprise the maize ODP2 promoter sequences set forth in SEQ ID NOs:1-3 and variants and fragments thereof. Compositions also include expression cassettes and vectors comprising a promoter sequence of the invention operably linked to a nucleotide sequence of interest. Transformed plants, plant cells, and seeds having an expression cassette of the invention stably incorporated into their genomes are further provided.

The compositions of the invention find use in methods directed to expressing nucleotide sequences of interest in a plant or plant cell, particularly in a seed-preferred manner. The methods of the invention comprise introducing into a plant or plant cell an expression cassette comprising an ODP2 promoter sequence, or a variant or fragment thereof, operably linked to a nucleotide sequence of interest. In some embodiments, the methods are directed to selectively expressing a nucleotide sequence of interest in a plant embryo or seed. In this manner, the promoter sequences of the invention find use in controlling the expression of operably linked coding sequences in a seed-preferred manner.

Nucleotide sequences of interest will typically provide for a modification of the phenotype of the plant. Such modification includes modulating the production of an endogenous product, as to amount, relative distribution, or the like, or production of an exogenous expression product to provide for a novel function or product in the plant. For example, a nucleotide sequence that encodes a polypeptide that confers herbicide, salt, pathogen, or insect resistance is encompassed by the present invention. Other nucleotide sequences of interest include, for example, sequences that encode polypeptides involved in the regulation of embryonic development, tissue differentiation, and biosynthesis of lipids, carbohydrates, or proteins. Nucleotide sequences that encode polypeptides that alter the oil content in a seed or plant are also of interest.

### BRIEF DESCRIPTION OF THE DRAWINGS

FIG. 1 provides the nucleotide sequence of the Zea mays ODP2 promoter (SEQ ID NO:1). The ODP2 promoter has two RY elements located from base pairs 287 to 293 and 929 to 935. SEQ ID NO:1 further comprises an Sph element and a G-box element at base pairs 285-293 and 796-801, respectively.

FIG. 2 provides an alignment of SEQ ID NOs:1-3. These ODP2 promoter sequences were isolated from three different maize genotypes.

FIG. 3A shows Lynx MPSS results of the tissue distribution of ODP2 expression in maize. FIG. 3B shows Lynx MPSS results for the expression levels of ODP2 in developing maize embryos at specified days after pollination.

### DETAILED DESCRIPTION OF THE INVENTION

The present invention provides compositions and methods directed to novel nucleotide sequences for plant promoters, particularly promoters that target gene expression in a plant seed. Specifically, the compositions of the invention comprise promoters isolated from the maize ODP2 gene described in U.S. Provisional Application No. 60/541,122 entitled “Maize AP2 Domain Transcription Factor Zm-ODP2 (Ovule Development Protein 2) and Its Use,” filed Feb. 2, 2004.

As used herein, “nucleic acid” includes reference to a deoxyribonucleotide or ribonucleotide polymer in either single- or double-stranded form, and unless otherwise limited, encompasses known analogues (e.g., peptide nucleic acids) having the essential nature of natural nucleotides in that they hybridize to single-stranded nucleic acids in a manner similar to naturally occurring nucleotides.

The invention encompasses isolated or substantially purified nucleic acid compositions. An “isolated” or “purified” nucleic acid molecule is substantially or essentially free from components that normally accompany or interact with the nucleic acid molecule or protein as found in its naturally occurring environment. Thus, an isolated or purified nucleic acid molecule is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived.

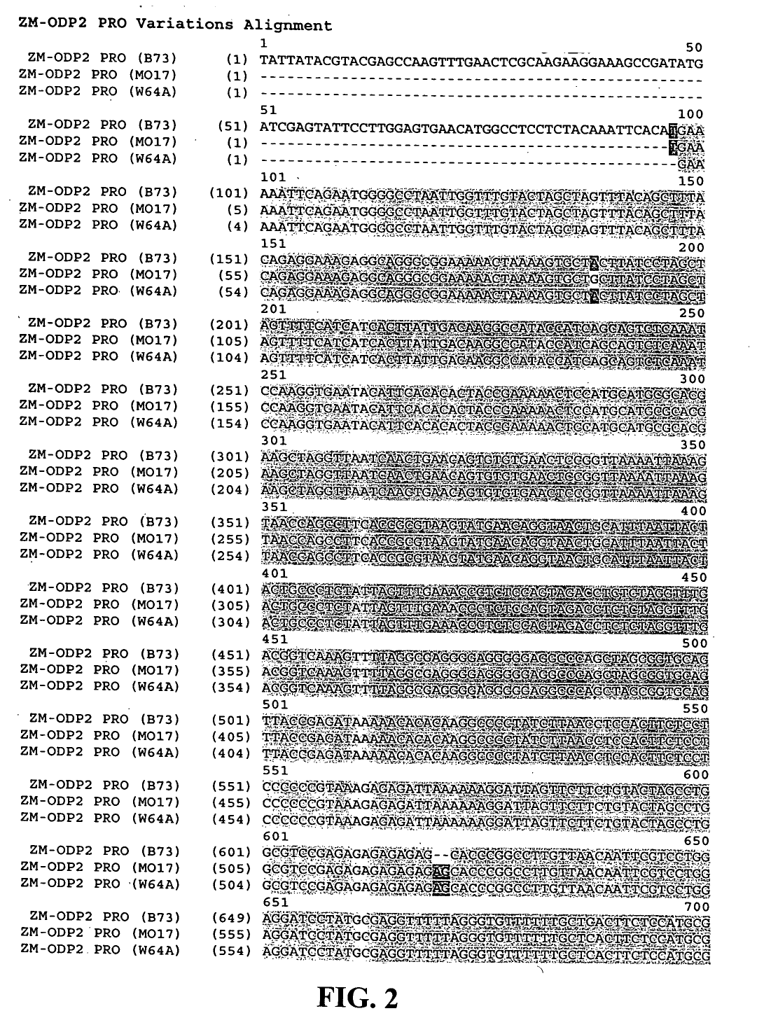

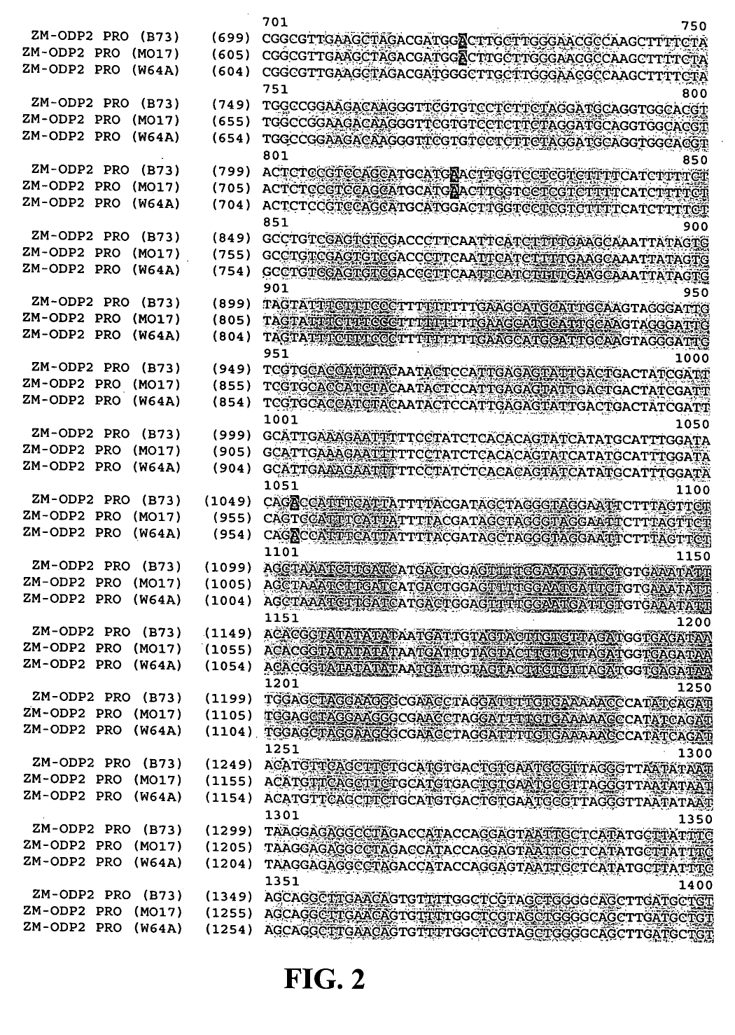

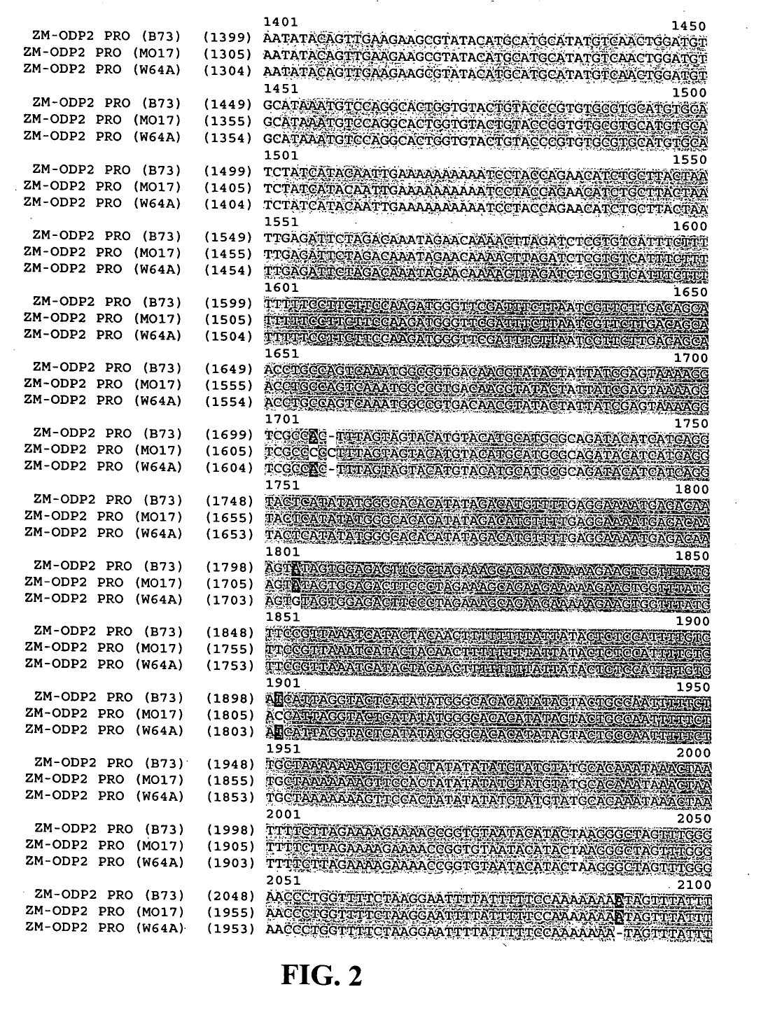

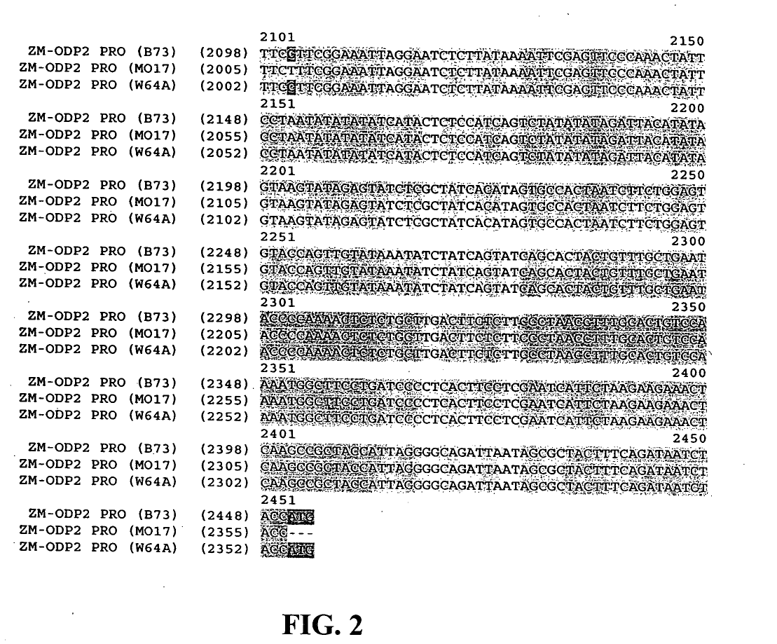

The compositions of the invention include isolated nucleic acid molecules comprising the promoter nucleotide sequences set forth in SEQ ID NOs:1-3 and variants and fragments thereof, as defined herein below. An alignment of SEQ ID NOs:1-3 is provided in FIG. 2. By “promoter” is intended a regulatory region of DNA usually comprising a TATA box capable of directing RNA polymerase II to initiate RNA synthesis at the appropriate transcription initiation site for a particular coding sequence. A promoter may additionally comprise other recognition sequences generally positioned upstream or 5′ to the TATA box, referred to as upstream promoter elements, which influence the transcription initiation rate. It is recognized that having identified the nucleotide sequences for the promoter regions disclosed herein, it is within the state of the art to isolate and identify further regulatory elements in the 5′ untranslated region upstream from the particular promoter regions identified herein. Thus, for example, the promoter regions disclosed herein may further comprise upstream regulatory elements such as those responsible for tissue and temporal expression of the coding sequence, enhancers, and the like. See particularly Australian Patent No. AU-A-77751/94 and U.S. Pat. Nos. 5,466,785 and 5,635,618. In the same manner, the promoter elements that enable expression in the desired tissue such as the plant embryo or seed, can be identified, isolated, and used with other core promoters to confer seed-preferred expression. By “core promoter” is intended a promoter that contains the essential nucleotide sequences for expression of an operably linked nucleotide sequence, and includes the TATA box and start of transcription. By this definition, a core promoter may or may not have detectable activity in the absence of specific sequences that may enhance the activity or confer tissue specific activity. A “plant promoter” is any promoter that drives expression in a plant or plant cell of the invention.

The maize ODP2 promoter sequences of the present invention, when assembled within a nucleotide construct such that the promoter is operably linked to a nucleotide sequence of interest, enables expression of the operably linked nucleotide sequence in a plant or plant cell. By “operably linked” is intended that the transcription or translation of the nucleotide sequence of interest is under the influence of the promoter sequence. In this manner, the nucleotide sequences for the promoters of the invention are provided in expression cassettes along with the nucleotide sequence of interest, typically a heterologous nucleotide sequence, for expression in the plant of interest. By “heterologous nucleotide sequence” is intended a sequence that is not naturally operably linked with the promoter sequence. While this nucleotide sequence is heterologous to the promoter sequence, it may be homologous, or native, or heterologous, or foreign, to the plant host.

It is recognized that the ODP2 promoter sequences of the invention may also be used with a native ODP2 coding sequence to genetically engineer plants having an altered phenotype. A nucleotide construct comprising an ODP2 promoter operably linked with its native ODP2 coding sequence may be used to transform any plant of interest to bring about a change in phenotype. Where the promoter and its native coding sequence are naturally occurring within a plant (i.e., in maize), transformation of the plant with these operably linked sequences results in a change in phenoype or insertion of these operably linked sequences within a different region of the chromosomes thereby altering the plant's genome. In other embodiments, an ODP2 promoter of the invention is operably linked to a nucleotide sequence that encodes an Oryza sativa ovule developmental protein, including, for example, OsAnt (Accession No. BAB89946) or OsBNM (Accession No. AAL47205). The nucleotide sequences encoding rice OsAnt and OsBNM are disclosed in U.S. Provisional Application No. 60/541,122 entitled “Maize AP2 Domain Transcription Factor Zm-ODP2 (Ovule Development Protein 2) and Its Use,” filed Feb. 2, 2004.

The use of the terms “polynucleotide constructs” or “nucleotide constructs” herein is not intended to limit the present invention to nucleotide constructs comprising DNA. Those of ordinary skill in the art will recognize that nucleotide constructs, particularly polynucleotides and oligonucleotides composed of ribonucleotides and combinations of ribonucleotides and deoxyribonucleotides, may also be employed in the methods disclosed herein. The nucleotide constructs, nucleic acids, and nucleotide sequences of the invention additionally encompass all complementary forms of such constructs, molecules, and sequences. Further, the nucleotide constructs, nucleotide molecules, and nucleotide sequences of the present invention encompass all nucleotide constructs, molecules, and sequences which can be employed in the methods of the present invention for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs, nucleic acids, and nucleotide sequences of the invention also encompass all forms of nucleotide constructs including, but not limited to, single-stranded forms, double-stranded forms, hairpins, stem-and-loop structures, and the like.

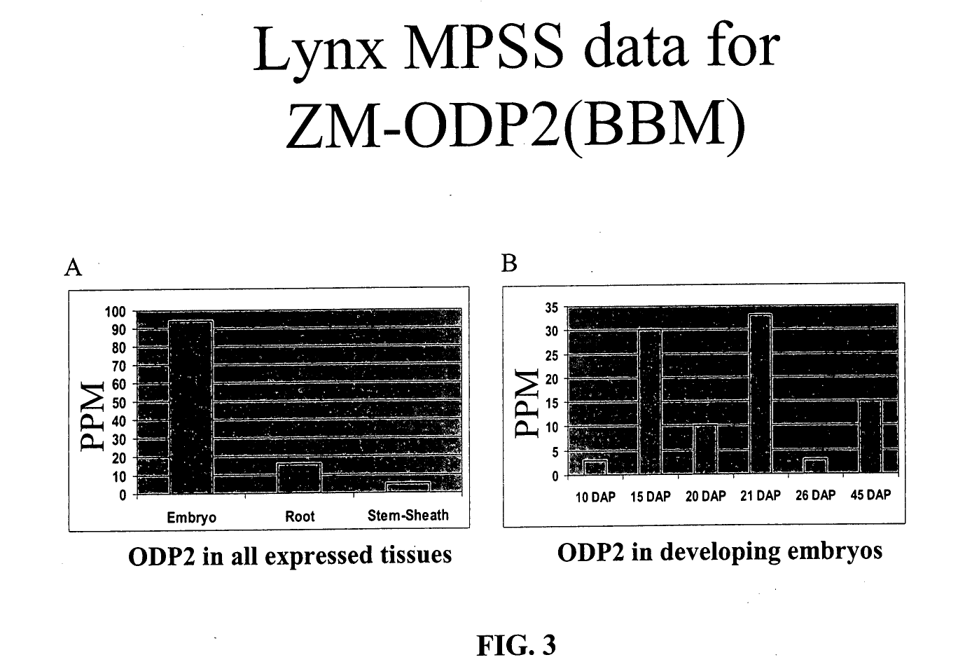

The ODP2 promoters of the present invention were isolated from the maize ODP2 gene described in U.S. Provisional Application No. 60/541,122 entitled “Maize AP2 Domain Transcription Factor Zm-ODP2 (Ovule Development Protein 2) and Its Use,” filed Feb. 2, 2004. The ODP2 protein is homologous to a number of polypeptides in the AP2 family of putative transcription factors. ODP2 also shares significant homology with the Arabidopsis Baby Boom polypeptide (AtBBM), an AP2 domain transcription factor. The AtBBM polypeptide has been shown to trigger formation of somatic embryos and cotyledon-like structures on seedlings and to activate signal transduction pathways leading to the induction of embryo development from differentiated somatic cells. See, for example, Boutilier et al. (2002) Plant Cell 14:1737-49), herein incorporated by reference. Furthermore, both ODP2 and AtBBM proteins are preferentially expressed in the developing embryo. See FIG. 3. Thus, the ODP2 promoter likely drives expression in a seed-preferred manner.

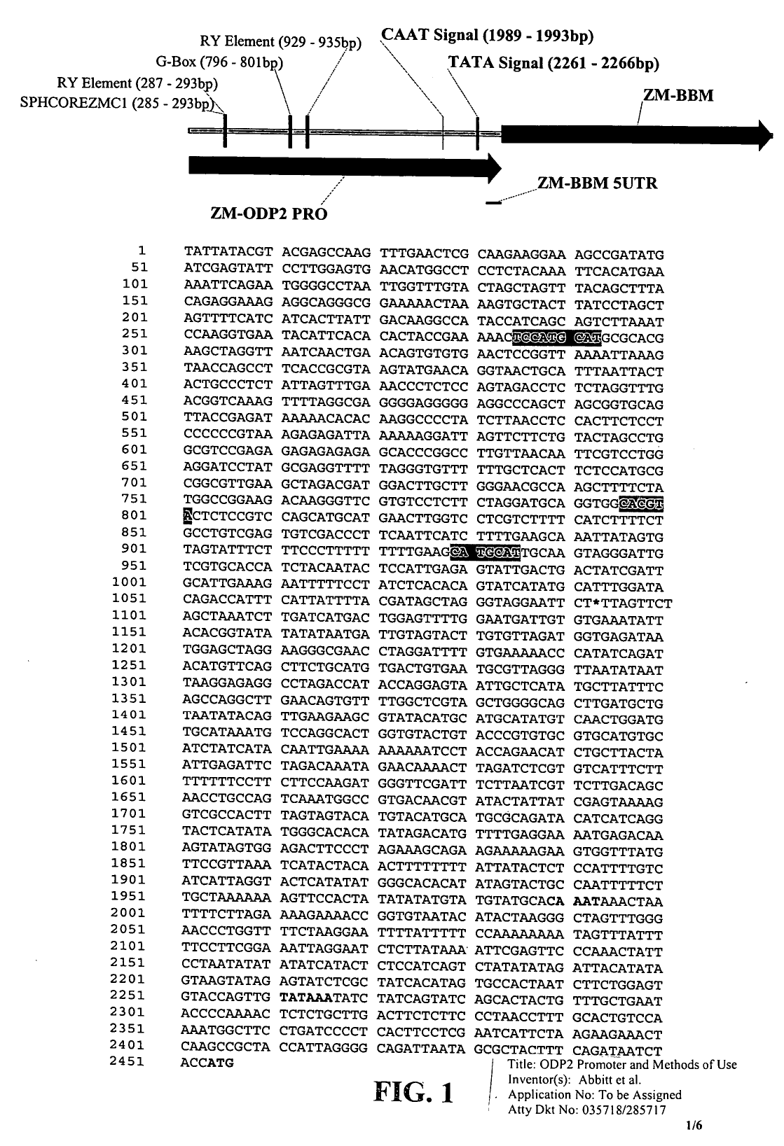

The ODP2 promoter sequence of SEQ ID NO:1 contains regulatory elements that further suggest that this promoter may drive seed-preferred expression in a plant. The ODP2 promoter disclosed herein comprises two RY elements (base pairs 287-293 and 929-935) and an Sph element (base pairs 285-293). See FIG. 1. RY and Sph elements are highly conserved among seed-specific promoters from both monocots and dicots. See, for example, Bobb et al. (1997) Nucleic Acids Research 25:641-647. Moreover, an RY element within the legumin box has been shown to play an important role in regulating seed-specific expression (Lelievre et al. (1992) Plant Physiol. 98:387-391). The promoter sequence of SEQ ID NO:1 further comprises a G-box element (base pairs 796-801). See FIG. 1. G-box or G-box related motifs have been identified in the promoters of a diverse set of unrelated genes and have further been shown to confer seed-specific expression in transgenic tobacco plants. See, for example, Salinas et al. (1992) The Plant Cell 4:1485-1493; Ouwerkerk et al. (1999) Mol. Gen. Genetics 261:635-643. Therefore, the OPD2 promoter sequences of the invention may find use in the expression of an operably linked nucleotide sequence of interest in a plant or plant cell. More specifically, the promoter sequences disclosed herein find use in selectively expressing a nucleotide sequence in a plant seed.

The promoter sequences of the invention can be operably linked to a nucleotide sequence of interest and stably incorporated into a plant or plant cell to drive seed-preferred expression of the operably linked nucleotide sequence. By “seed-preferred expression” is intended favored expression in the seed, including but not limited to, at least one of embryo, zygote, kernel, pericarp, endosperm, nucellus, aleurone, pedicel, and the like. While some level of expression of the nucleotide sequence of interest may occur in other tissue types, expression occurs most abundantly in the seed, as defined herein above.

Modifications of the isolated promoter sequences of the present invention can provide for a range of expression levels of the heterologous nucleotide sequence. Thus, they may be modified to be weak promoters or strong promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts.

Fragments and variants of the disclosed promoter sequence are also encompassed by the present invention. By “fragment” is intended a portion of the promoter sequence. Fragments of a promoter sequence may retain biological activity and hence encompass fragments capable of driving seed-preferred expression of an operably linked nucleotide sequence. Thus, for example, less than the entire promoter sequence disclosed herein may be utilized to drive expression of an operably linked nucleotide sequence of interest, such as a nucleotide sequence encoding a heterologous protein. It is within skill in the art to determine whether such fragments decrease expression levels or alter the nature of expression, i.e., constitutive, inducible, or tissue-preferred expression. Alternatively, fragments of a promoter nucleotide sequence that are useful as hybridization probes, such as described below, generally do not retain this regulatory activity. Thus, fragments of a nucleotide sequence may range from at least about 20 nucleotides, about 50 nucleotides, about 100 nucleotides, and up to the full-length nucleotide sequence of the invention.

As used herein, “full-length sequence” in reference to a specified polynucleotide means having the entire nucleic acid sequence of a native sequence. By “native sequence” is intended an endogenous sequence, i.e., a non-engineered sequence found in an organism's genome.

Thus, a fragment of an ODP2 promoter nucleotide sequence may encode a biologically active portion of the ODP2 promoter or it may be a fragment that can be used as a hybridization probe or PCR primer using methods disclosed below. A biologically active portion of an ODP2 promoter can be prepared by isolating a portion of one of the ODP2 promoter nucleotide sequences of the invention and assessing the activity of that portion of the ODP2 promoter. Nucleic acid molecules that are fragments of a promoter nucleotide sequence comprise at least 15, 20, 25, 30, 35, 40, 45, 50, 75, 100, 325, 350, 375, 400, 425, 450, 500, 550, 600, 650, 700, 800, 900, 1200, 1500, 1800, 2000, 2200, 2400 contiguous nucleotides, or up to the number of nucleotides present in the full-length promoter nucleotide sequence disclosed herein.

The nucleotides of such fragments will usually comprise the TATA recognition sequence of the particular promoter sequence. In some embodiments, fragments of an ODP2 promoter will further comprise the regulatory regions described herein above, i.e., RY, Sph, and/or G-box elements. Such fragments may be obtained by use of restriction enzymes to cleave the naturally occurring promoter nucleotide sequence disclosed herein; by synthesizing a nucleotide sequence from the naturally occurring sequence of the promoter DNA sequence; or may be obtained through the use of PCR technology. See particularly, Mullis et al. (1987) Methods Enzymol. 155:335-350, and Erlich, ed. (1989) PCR Technology (Stockton Press, New York). Variants of these promoter fragments, such as those resulting from site-directed mutagenesis, are also encompassed by the compositions of the present invention.

By “variants” is intended sequences having substantial similarity with a promoter sequence disclosed herein. For nucleotide sequences, naturally occurring variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis. Generally, variants of a particular nucleotide sequence of the invention will have at least 40%, 50%, 60%, 65%, 70%, generally at least 75%, 80%, 85%, preferably about 90%, 91%, 92%, 93%, 94%, to 95%, 96%, 97%, and more preferably about 98%, 99% or more sequence identity to that particular nucleotide sequence as determined by sequence alignment programs described elsewhere herein using default parameters. Biologically active variants are also encompassed by the present invention. Biologically active variants include, for example, the native promoter sequence of the invention having one or more nucleotide substitutions, deletions, or insertions.

Promoter activity for any of the ODP2 promoter variants or fragments of the invention may be assayed using a variety of techniques well known to one of ordinary skill in the art, including, for example, Northern blot analysis, reporter activity measurements taken from transcriptional fusions, and the like. See, for example, Sambrook et al. (1989) Molecular Cloning: A Laboratory Manual (2d ed., Cold Spring Harbor Laboratory Press, Plainview, N.Y.), herein incorporated by reference. Alternatively, promoter assays may be based on the measurement of levels of a reporter gene such as green fluorescent protein (GFP) or the like produced under the control of a promoter fragment or variant. See, for example, U.S. Pat. No. 6,072,050, herein incorporated by reference. Variants, fragments, or other nucleotide sequences of the invention may be routinely assayed for activity using such assays; for example, large collections of randomly generated fragments may be quickly and routinely screened for promoter activity using these or other methods. In the instant case, for example, such assays might include the use of the promoters of the invention to drive the expression of the GUS reporter gene, as well as the cytokinin producing gene, isopentenyl transferase (IPT).

Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein.

The promoter sequences of the invention can be used to isolate corresponding sequences from other organisms, particularly other plants, more particularly other monocots. In this manner, methods such as PCR, hybridization, and the like can be used to identify such sequences based on their sequence homology to the sequence set forth herein. Sequences isolated based on their sequence identity to the entire ODP2 promoter sequence set forth herein or to fragments thereof are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the ODP2 promoter sequence of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, an entire ODP2 promoter sequence disclosed herein, or one or more portions thereof, may be used as a probe capable of specifically hybridizing to corresponding ODP2 promoter sequences. To achieve specific hybridization under a variety of conditions, such probes include sequences that are unique among ODP2 promoter sequences and are preferably at least about 10 nucleotides in length, and most preferably at least about 20 nucleotides in length. Such probes may be used to amplify corresponding ODP2 promoter sequences from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

Thus, isolated sequences that have seed-preferred promoter activity and which hybridize under stringent conditions to an ODP2 promoter sequence disclosed herein, or to fragments thereof, are encompassed by the present invention. The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70% sequence identity, preferably at least 80%, more preferably at least 90%, and most preferably at least 95%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C. lower than the Tₘ, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman and Wunsch (1970) J. Mol. Biol. 48:443-453. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

In some embodiments, expression cassettes comprising an ODP2 promoter, or a variant or fragment thereof, operably linked to a nucleotide sequence of interest are provided for expression in a plant of interest. The operably linked nucleotide sequence of interest may be any sequence whose expression in a plant or plant cell is desirable. Nucleotide sequences whose selective expression in a plant seed is desirable are of particular interest. The nucleotide sequence of interest will typically be a heterologous nucleotide sequence, as defined herein above. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

The expression cassettes and vectors of the invention find use in expressing a nucleotide sequence of interest in a plant or plant cell. In preferred embodiments, methods for expressing a nucleotide sequence of interest in a tissue-preferred manner, more preferably in a seed-preferred manner are provided. An expression cassette of the invention is provided with a plurality of restriction sites for insertion of the nucleotide sequence of interest to be under the transcriptional regulation of the regulatory regions (i.e., promoter). The expression cassette may additionally contain selectable marker genes. In particular embodiments, the expression cassette is transferred to a vector for expression of the nucleotide sequence of interest in a plant or plant cell. Vectors for delivery of nucleotide constructs to a variety of plants and plant cells are well known in the art. Plant cells, plants, and seeds thereof, having an expression cassette of the invention stably incorporated into their genome are further provided.

As used herein, “vector” refers to a DNA molecule such as a plasmid, cosmid, or bacterial phage for introducing a nucleotide construct, for example, an expression cassette, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance, or ampicillin resistance.

An expression cassette of the invention will include in the 5′-3′ direction of transcription, a transcriptional and translational initiation region (i.e., an ODP2 promoter or a variant or fragment thereof), a nucleotide sequence of interest, and a transcriptional and translational termination region (i.e., termination region) functional in plants. The promoter may be native or analogous, or foreign or heterologous, to the plant host and/or to the nucleotide sequence of interest. Additionally, the promoter may be the natural sequence or alternatively a synthetic sequence. Where the promoter is “foreign” or “heterologous” to the plant host, it is intended that the promoter is not found in the native plant into which the promoter is introduced. Where the promoter is “foreign” or “heterologous” to the nucleotide sequence of interest, it is intended that the promoter is not the native or naturally occurring promoter for the operably linked nucleotide sequence.

The termination region may be native with the transcriptional initiation region comprising the promoter nucleotide sequence of the present invention, may be native with the nucleotide sequence of interest, or may be derived from another source. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, the nucleic acid molecules of the invention may be optimized for increased expression in the transformed plant. That is, a sequence can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

It is recognized that to increase transcription levels enhancers may be utilized in combination with the promoter regions of the invention. Enhancers are nucleotide sequences that act to increase the expression of a promoter region. Enhancers are known in the art and include the SV40 enhancer region, the 35S enhancer element, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10: 143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

The regulatory elements of the invention can also be used for callus-preferred expression of selectable markers. The regulatory elements of the present invention operably linked to a herbicide resistant gene would allow plants to be regenerated that have no field resistance to herbicide but may be completely resistant to the herbicide in the callus stage. Callus-preferred expression would allow selection of the transformant but would not require the plant to express the transgene in the field, thereby maintaining or even improving yield.

Selectable marker genes for selection of transformed cells or tissues can be included in the transformation vectors. These can include genes that confer antibiotic resistance or resistance to herbicides. Examples of suitable selectable marker genes include, but are not limited to: genes encoding resistance to chloramphenicol, Herrera Estrella et al. (1983) EMBO J. 2:987-992; methotrexate, Herrera Estrella et al. (1983) Nature 303:209-213; Meijer et al. (1991) Plant Mol. Biol. 16:807-820; hygromycin, Waldron et al. (1985) Plant Mol. Biol. 5:103-108; Zhijian et al. (1995) Plant Science 108:219-227; streptomycin, Jones et al. (1987) Mol. Gen. Genet. 210:86-91; spectinomycin, Bretagne-Sagnard et al. (1996) Transgenic Res. 5:131-137; bleomycin, Hille et al. (1990) Plant Mol. Biol. 7:171-176; sulfonamide, Guerineau et al. (1990) Plant Mol. Biol. 15:127-136; bromoxynil, Stalker et al. (1988) Science 242:419-423; glyphosate, Shaw et al. (1986) Science 233:478-481; phosphinothricin, DeBlock et al. (1987) EMBO J. 6:2513-2518.

Other genes that could serve utility in the recovery of transgenic events but might not be required in the final product would include, but are not limited to: GUS (β-glucoronidase), Jefferson (1987) Plant Mol. Biol. Rep. 5:387); GFP (green florescence protein), Chalfie et al. (1994) Science 263:802; luciferase, Teeri et al. (1989) EMBO J. 8:343; and the maize genes encoding for anthocyanin production, Ludwig et al. (1990) Science 247:449.

The promoter nucleotide sequences and methods disclosed herein are useful in regulating expression, particularly seed-preferred expression, of any nucleotide sequence of interest in a host plant in order to vary the phenotype of a plant. Various changes in phenotype are of interest including modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Because the promoter sequences of the invention drive expression in a seed-preferred manner, nucleotide sequences whose selective expression in the embryo or seed is desirable are of particular interest. Such sequences include those that encode polypeptides involved in the regulation of embryonic development and tissue differentiation. Other heterologous nucleotide sequences of interest include genes involved in the modulation of the oil content in a seed or in the biosynthesis of lipids, carbohydrates, and proteins. In a particular embodiment, the nucleotide sequence of interest encodes the maize ODP2 protein. In other embodiments, the nucleotide sequence of interest encodes a rice ovule developmental protein, for example, OsAnt (Accession No. BAB89946) or OsBNM (Accession No. AAL47205).

Nucleotide sequences of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Nucleotide sequences of interest include, generally, those involved in oil, starch, carbohydrate, or nutrient metabolism as well as those affecting kernel size, sucrose loading, and the like.

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and WO 98/20133, the disclosures of which are herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference). Other agronomically important genes encode latex, Floury 2, growth factors, seed storage factors, and transcription factors.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); lectins (Van Damme et al. (1994) Plant Mol. Biol. 24:825); and the like.

Genes encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), genes coding for resistance to glyphosate (e.g., the EPSPS gene and the GAT gene; see, for example, U.S. Publication No. 20040082770 and WO 03/092360), or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802, and 5,990,389.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as Ketothiolase, PHBase (polyhydroxyburyrate synthase), and acetoacetyl-CoA reductase (see Schubert et al. (1988) J. Bacteriol. 170:5837-5847) facilitate expression of polyhyroxyalkanoates (PHAs).

Exogenous products include plant enzymes and products as well as those from other sources including procaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

In some embodiments, the activity of a target protein of interest is reduced or eliminated by transforming a plant or plant cell with an expression cassette comprising an ODP2 promoter sequence of the invention operably linked to a polynucleotide that inhibits the expression of the target protein. The polynucleotide may inhibit the expression of one or more target proteins directly, by preventing translation of the target protein messenger RNA, or indirectly, by encoding a polypeptide that inhibits the transcription or translation of a gene encoding the protein of interest. Methods for inhibiting or eliminating the expression of a gene in a plant are well known in the art, and any such method may be used in the present invention to inhibit the expression of one or more target proteins.

In accordance with the present invention, the expression of a target protein is inhibited if the protein level of the protein of interest is statistically lower than the protein level of the same protein in a plant that has not been genetically modified or mutagenized to inhibit the expression of that protein. In particular embodiments of the invention, the protein level of the target protein in a modified plant according to the invention is less than 95%, less than 90%, less than 80%, less than 70%, less than 60%, less than 50%, less than 40%, less than 30%, less than 20%, less than 10%, or less than 5% of the protein level of the same protein in a plant that is not a mutant or that has not been genetically modified to inhibit the expression of the protein of interest. The expression level of the target protein may be measured directly, for example, by assaying for its level of expression in the plant cell or plant, or indirectly, for example, by measuring the activity of the target protein in the plant cell or plant.

In other embodiments of the invention, the activity of one or more target proteins is reduced or eliminated by transforming a plant or plant cell with an expression cassette comprising an ODP2 promoter of the invention operably linked to a polynucleotide encoding a polypeptide that inhibits the activity of one or more target proteins of interest. The activity of a target protein is inhibited according to the present invention if the activity of the protein of interest is statistically lower than the activity of the same protein in a plant that has not been genetically modified to inhibit the activity of that protein. In particular embodiments of the invention, the activity of the target protein in a modified plant according to the invention is less than 95%, less than 90%, less than 80%, less than 70%, less than 60%, less than 50%, less than 40%, less than 30%, less than 20%, less than 10%, or less than 5% of the activity of the same protein in a plant that that has not been genetically modified to inhibit the expression of that target protein. The activity of a target protein is “eliminated” according to the invention when it is not detectable by standard assay methods.

In other embodiments, the activity of a target protein may be reduced or eliminated by disrupting the gene encoding the target protein. The invention encompasses mutagenized plants that carry mutations in target genes, where the mutations reduce expression of the target gene or inhibit the activity of the protein encoded by the target gene.

Thus, many methods may be used to reduce or eliminate the activity of a target protein of interest. More than one method may be used to reduce the activity of a single protein of interest. In addition, combinations of methods may be employed to reduce or eliminate the activity of two or more different proteins.

Non-limiting examples of methods of reducing or eliminating the expression of a target protein in a plant of interest are given below.

Reduction of the activity of specific genes (also known as gene silencing or gene suppression) is desirable for several aspects of genetic engineering in plants. Many techniques for gene silencing are well known to one of skill in the art, including, but not limited to, antisense technology (see, e.g., Sheehy et al. (1988) Proc. Natl. Acad. Sci. USA 85:8805-8809; and U.S. Pat. Nos. 5,107,065; 5,453,566; and 5,759,829); cosuppression (e.g., Taylor (1997) Plant Cell 9:1245; Jorgensen (1990) Trends Biotech. 8(12):340-344; Flavell (1994) Proc. Natl. Acad. Sci. USA 91:3490-3496; Finnegan et al. (1994) Bio/Technology 12:883-888; and Neuhuber et al. (1994) Mol. Gen. Genet. 244:230-241); RNA interference (Napoli et al. (1990) Plant Cell 2:279-289; U.S. Pat. No. 5,034,323; Sharp (1999) Genes Dev. 13:139-141; Zamore et al. (2000) Cell 101:25-33; and Montgomery et al. (1998) Proc. Natl. Acad. Sci. USA 95:15502-15507), virus-induced gene silencing (Burton et al. (2000) Plant Cell 12:691-705; and Baulcombe (1999) Curr. Op. Plant Bio. 2:109-113); target-RNA-specific ribozymes (Haseloff et al. (1988) Nature 334: 585-591); hairpin structures (Smith et al. (2000) Nature 407:319-320; WO 99/53050; WO 02/00904; and WO 98/53083); ribozymes (Steinecke et al. (1992) EMBO J. 11:1525; and Perriman et al. (1993) Antisense Res. Dev. 3:253); oligonucleotide-mediated targeted modification (e.g., WO 03/076574 and WO 99/25853); Zn-finger targeted molecules (e.g., WO 01/52620; WO 03/048345; and WO 00/42219); transposon tagging (Maes et al. (1999) Trends Plant Sci. 4:90-96; Dharmapuri and Sonti (1999) FEMS Microbiol. Lett. 179:53-59; Meissner et al. (2000) Plant J 22:265-274; Phogat et al. (2000) J Biosci. 25:57-63; Walbot (2000) Curr. Opin. Plant Biol. 2:103-107; Gai et al. (2000) Nucleic Acids Res. 28:94-96; Fitzmaurice et al. (1999) Genetics 153:1919-1928; Bensen et al. (1995) Plant Cell 7:75-84; Mena et al. (1996) Science 274:1537-1540; and U.S. Pat. No. 5,962,764); each of which is herein incorporated by reference; and other methods or combinations of the above methods known to those of skill in the art.

In some embodiments, the ODP2 promoters of the invention find use in inducing early plant embryo abortion. In a particular embodiment, an ODP2 promoter is used in conjunction with the components of the ecdysone-inducible system to produce an embryo-specific expression system. See, for example, Martinez et al. (1999) Mol. Gen. Genet. 261:546-552; Martinez et al. (1999) Plant J 19:97-106; Padidam et al. (2003) Transgenic Res. 12:101-109, which describe the ecdysone-inducible system in plants. This embryo-specific expression system can be used to drive expression of a cytotoxic gene, such as, for example, dam methylase. Induction of early plant embryo abortion has potential applications for apomixis and for food and feed purposes (e.g., making endosperm only seed).

In a further embodiment, early plant embryo abortion is induced by expressing the c-terminal (dam-c) and n-terminal (dam-n) half of dam methylase from different cassettes fused to split intein fragments (the c-terminal half and n-terminal half, respectively). See, for example, Yang et al. (2003) Proc. Natl. Acad. Sci. 100:3513-3518, which describes using intein splicing to express a transgene in plants. When these two fusion-genes are expressed in the same cell, protein trans-splicing occurs to produce a mature functional protein, in this case dam methylase. Two constructs are made, an ODP2:dam-n and an ODP2:dam-c. Each of these constructs is transformed into one of the parent inbreds used to ultimately make a hybrid plant. Expressed separately in this fashion, neither half is functional. However, when the two plants are crossed to produce the hybrid embryo (and endosperm), functional cytotoxic dam methylase is produced in the embryo, and an embryoless seed results.

The terms “polypeptide,” “peptide,” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

As used herein, the terms “encoding” or “encoded” when used in the context of a specified nucleic acid mean that the nucleic acid comprises the requisite information to direct translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

As used herein, the term “plant” includes reference to whole plants, plant organs (e.g., leaves, stems, roots, etc.), seeds, plant cells, and progeny of same. Parts of transgenic plants are to be understood within the scope of the invention to comprise, for example, plant cells, protoplasts, tissues, callus, embryos as well as flowers, ovules, stems, fruits, leaves, roots originating in transgenic plants or their progeny previously transformed with a DNA molecule of the invention and therefore consisting at least in part of transgenic cells, are also an object of the present invention.

As used herein, the term “plant cell” includes, without limitation, seeds suspension cultures, embryos, meristematic regions, callus tissue, leaves, roots, shoots, gametophytes, sporophytes, pollen, and microspores. The class of plants that can be used in the methods of the invention is generally as broad as the class of higher plants amenable to transformation techniques, including both monocotyledonous and dicotyledonous plants. Such plants include, for example, Solanum tuberosum and Zea mays.

The present invention may be used for the introduction of a nucleotide sequence into any plant species, including, but not limited to, monocots and dicots. In this manner, genetically modified plants, plant cells, plant tissue, seed, root, and the like can be obtained. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Preferably, plants of the present invention are crop plants (for example, corn, alfalfa, sunflower, Brassica, soybean, cotton, safflower, peanut, sorghum, wheat, millet, tobacco, etc.), more preferably corn and soybean plants, yet more preferably corn plants.

Additional plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

The methods of the invention involve introducing a nucleotide construct into a plant or plant cell. By “introducing” is intended presenting to the plant the nucleotide construct in such a manner that the construct gains access to the interior of a cell of the plant. The methods of the invention do not depend on a particular method for introducing a nucleotide construct to a plant, only that the nucleotide construct gains access to the interior of at least one cell of the plant. Methods for introducing nucleotide constructs into plants are known in the art including, but not limited to, stable transformation methods, transient transformation methods, and virus-mediated methods.

By “stable transformation” is intended that the nucleotide construct introduced into a plant integrates into the genome of the plant and is capable of being inherited by progeny thereof. By “transient transformation” is intended that a nucleotide construct introduced into a plant does not integrate into the genome of the plant.

The nucleotide constructs of the invention may be introduced into plants by contacting plants with a virus or viral nucleic acids. Generally, such methods involve incorporating a nucleotide construct of the invention within a viral DNA or RNA molecule. Methods for introducing nucleotide constructs into plants and expressing a protein encoded therein, involving viral DNA or RNA molecules, are known in the art. See, for example, U.S. Pat. Nos. 5,889,191, 5,889,190, 5,866,785, 5,589,367, and 5,316,931; herein incorporated by reference.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lecl transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The cells that have been transformed may be grown into plants in accordance with conventional ways. See, for example, McConnick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

As discussed above, the maize ODP2 gene is preferentially expressed in the embryo. See FIG. 3A. Based on the LYNX expression data shown in FIG. 3B, ODP2 expression likely occurs at 10-45 DAP (days after pollination) in embryo development. Therefore, ODP2 expression is a marker for embryogenesis. Embryo-specific expression of ODP2 is regulated by an operably linked ODP2 promoter in vivo, for example, the nucleotide sequences disclosed herein above. Thus, increased activity of an ODP2 promoter can also serve as a marker for entry of a plant cell into embryonic development.

The ODP2 promoter sequences of the invention find further use in defining culture conditions that alter the embryo-forming capacity of a tissue in vitro. In this embodiment, the activity of the ODP2 promoter is monitored under various in vitro culture conditions and increased activity of the ODP2 promoter serves as an indicator of embryogenesis. Therefore, culture conditions that facilitate or enhance the formation of embryogenic cells are identified on the basis of ODP2 promoter activity within the cultured tissue. Various methods can be employed to monitor the activity of the ODP2 promoter. For example, increased mRNA or polypeptide levels of the native ODP2 gene can be assayed. In other embodiments, an expression cassette comprising the ODP2 promoter of the invention operably linked to a reporter gene is introduced into a plant or plant cell of interest, and expression of the reporter gene is monitored. Reporter genes of interest include, for example, GUS (Jefferson et al. (1987) EMBO J. 6:3901-3907), luciferase (Riggs et al. (1987) Nucleic Acids Res. 15(19):8115 and Luehrsen et al. (1992) Methods Enzymol. 216:397-414), and GFP (Haseloff and Amos (1995) Trends Genet. 11:328-329).

The invention further provides compositions for screening compounds that modulate expression in the plant embryo or seed. The vectors, cells, and plants disclosed herein can be used for screening candidate molecules for agonists and antagonists of the ODP2 promoter. For example, a reporter gene can be operably linked to an ODP2 promoter and expressed as a transgene in a plant. Compounds to be tested are then added, and reporter gene expression is measured to determine the effect on promoter activity.

The article “a” and “an” are used herein to refer to one or more than one (i.e., to at least one) of the grammatical object of the article. By way of example, “an element” means one or more element.

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The above-defined terms are more fully defined by reference to the specification as a whole.

The following examples are offered by way of illustration and not by way of limitation.

### EXPERIMENTAL

### Example 1

### Isolation of the ODP2 Promoter

Sequences overlapping the 5′ end of the ODP2 transcript were obtained by BLASTing against the Genome Survey Sequence (GSS) Dataset in the NCBI database. This provided us with 2453 bp of sequence upstream of the start codon. Based on that sequence, three sets of PCR primers were created to verify that the sequence was overlapping with the ODP2 transcript in maize (B73) genomic DNA using the High Fidelity Polymerase Supermix from Invitrogen. The amplifications products were isolated and cloned into pGEMT-easy (Invitrogen) and sequenced.

Verification Primers

Product of length 1011 bp

| | |
|---|---|
| TGTACATGCATGCGCAGATA | (SEQ ID NO:4) |
| CAAACTGTGCTGCTAGTGCT | (SEQ ID NO:5) |

Product of length 1810 bp

| | |
|---|---|
| TTTGAAGCATGCATTGCAAG | (SEQ ID NO:6) |
| CAAACTGTGCTGCTAGTGCT | (SEQ ID NO:5) |

Product of length 2635 bp

| | |
|---|---|
| TGAAAAATTCAGAATGGGGC | (SEQ ID NO:7) |
| CAAACTGTGCTGCTAGTGCT | (SEQ ID NO:5) |

The PCR results were positive, indicating that the sequence was upstream of the transcript. To isolate cloning versions of the promoter, additional PCR primers were ordered. These primers contain cloning sites to facilitate vector construction. The nonhomologous regions of the Primers are indicated with an underline. These regions contain various cloning sites. The 1385 bp version eliminates the RY-GBOX-RY motif. Both versions were isolated from maize (B73) genomic DNA using the High Fidelity Polymerase Supermix from Invitrogen. The amplifications products were isolated and cloned into pGEMT-easy (Invitrogen) and sequenced.

ZM-ODP2 PRO PCR Primers

Product of length 2477 bp (ZM-ODP2 PRO)

(SEQ ID NO:8)

GGTTACCCGGACCGGAGCTCTATTATACGTACGAGCCAAG

(SEQ ID NO:9)

CCATGGTAGATTATCTGAAAGTAGCGCTATTAATCTGCCCCT
AATGGTAGCG

Product of length 1385 bp (ZM-ODP2A PRO)

(SEQ ID NO:10)

GGTTACCCGGACCGGAATTCTTAGTTCTAGCTAAATCTTG

(SEQ ID NO:9)

CCATGGTAGATTATCTGAAAGTAGCGCTATTAATCTGCCCCT
AATGGTAGCG

### Example 2

### Transformation of Maize and Regeneration of Transgenic Plants

Immature maize embryos from greenhouse donor plants are bombarded with a plasmid containing the ODP2 promoter (SEQ ID NO:1) operably linked to a nucleotide sequence of interest and the selectable marker gene PAT (Wohlleben et al. (1988) Gene 70:25-37), which confers resistance to the herbicide Bialaphos. Alternatively, the selectable marker gene is provided on a separate plasmid. Transformation is performed as follows. Media recipes follow below.

Preparation of Target Tissue

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA

A plasmid vector comprising the ODP2 promoter (SEQ ID NO:1) operably linked to a nucleotide sequence of interest is made. This plasmid DNA plus plasmid DNA containing a PAT selectable marker is precipitated onto 1.1 μm (average diameter) tungsten pellets using a CaCl₂ precipitation procedure as follows:
100 μl prepared tungsten particles in water
10 μl (1 μg) DNA in Tris EDTA buffer (1 μg total DNA)
100 μl 2.5 M CaCl₂
10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for expression of the nucleotide sequence of interest by assays known in the art, such as, for example, immunoassays and western blotting.

Bombardment and Culture Media

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### Example 3

### Agrobacterium-Mediated Transformation of Maize and Regeneration of Transgenic Plants

For Agrobacterium-mediated transformation of maize with a polynucleotide construct comprising the ODP2 promoter (SEQ ID NO:1) operably linked to a nucleotide sequence of interest, preferably the method of Zhao is employed (U.S. Pat. No. 5,981,840, and PCT patent publication WO98/32326; the contents of which are hereby incorporated by reference). Briefly, immature embryos are isolated from maize and the embryos contacted with a suspension of Agrobacterium, where the bacteria are capable of transferring the polynucleotide construct to at least one cell of at least one of the immature embryos (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step), and preferably calli grown on selective medium are cultured on solid medium to regenerate the plants.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. An isolated nucleic acid molecule comprising a nucleotide sequence selected from the group consisting of:
a) a nucleotide sequence comprising the sequence set forth in SEQ ID NO:1, 2, or 3;
b) a nucleotide sequence comprising at least 20 contiguous nucleotides of the sequence set forth in SEQ ID NO:1, 2, or 3, wherein said nucleotide sequence initiates transcription in a plant cell;
c) a nucleotide sequence comprising a sequence having at least 70% sequence identity to the sequence set forth in SEQ ID NO:1, 2, or 3, wherein said nucleotide sequence initiates transcription in a plant cell; and,
d) a nucleotide sequence that hybridizes under stringent conditions to a complement of a nucleotide sequence of a), wherein said nucleotide sequence initiates transcription in a plant cell.

2. An expression cassette comprising a nucleotide sequence of claim 1 operably linked to a heterologous nucleotide sequence of interest.

3. A vector comprising the expression cassette of claim 2.

4. A plant cell having stably incorporated into its genome the expression cassette of claim 2.

5. A plant having stably incorporated into its genome the expression cassette of claim 2.

6. The plant of claim 5, wherein said plant is a monocot.

7. The plant of claim 6, wherein said monocot is maize.

8. The plant of claim 5, wherein said plant is a dicot.

9. A transformed seed of the plant of claim 5.

10. A method for expressing a nucleotide sequence in a plant, said method comprising introducing into a plant an expression cassette, said expression cassette comprising a promoter operably linked to a heterologous nucleotide sequence of interest, wherein said promoter comprises a nucleotide sequence selected from the group consisting of:
a) a nucleotide sequence comprising the sequence set forth in SEQ ID NO:1, 2, or 3;
b) a nucleotide sequence comprising at least 20 contiguous nucleotides of the sequence set forth in SEQ ID NO:1, 2, or 3, wherein said nucleotide sequence initiates transcription in a plant cell;
c) a nucleotide sequence comprising a sequence having at least 70% sequence identity to the sequence set forth in SEQ ID NO:1, 2, or 3, wherein said nucleotide sequence initiates transcription in a plant cell; and,
d) a nucleotide sequence that hybridizes under stringent conditions to a complement of a nucleotide sequence of a), wherein said nucleotide sequence initiates transcription in a plant cell.

11. The method of claim 10, wherein said heterologous nucleotide sequence of interest is selectively expressed in a plant seed.

12. The method of claim 10, wherein said heterologous nucleotide sequence of interest encodes a polypeptide that confers herbicide, salt, pathogen, or insect resistance.

13. The method of claim 10, wherein said heterologous nucleotide sequence of interest encodes a polypeptide involved in biosynthesis of lipids, carbohydrates, or proteins.

14. The method of claim 10, wherein said heterologous nucleotide sequence of interest encodes a polypeptide involved in regulation of embryonic development.

15. The method of claim 10, wherein said heterologous nucleotide sequence of interest encodes a polypeptide involved in regulation of tissue differentiation.

16. The method of claim 10, wherein said heterologous nucleotide sequence of interest encodes a polypeptide that modulates oil content.

17. A method for selectively expressing a nucleotide sequence in a plant seed, said method comprising introducing into a plant cell an expression cassette, said expression cassette comprising a promoter operably linked to a heterologous nucleotide sequence of interest, wherein said promoter comprises a nucleotide sequence selected from the group consisting of:
a) a nucleotide sequence comprising the sequence set forth in SEQ ID NO:1, 2, or 3;
b) a nucleotide sequence comprising at least 20 contiguous nucleotides of the sequence set forth in SEQ ID NO:1, 2, or 3, wherein said nucleotide sequence initiates transcription in a plant cell;
c) a nucleotide sequence comprising a sequence having at least 70% sequence identity to the sequence set forth in SEQ ID NO:1, 2, or 3, wherein said nucleotide sequence initiates transcription in a plant cell; and,
d) a nucleotide sequence that hybridizes under stringent conditions to a complement of a nucleotide sequence of a), wherein said nucleotide sequence initiates transcription in a plant cell.
